(19) United States
(12) Patent Application Publication
(10) Pub. No.: US 2024/0207785 A1
(43) Pub. Date: Jun. 27, 2024

(54) PROCESS TO CONTINUOUSLY TREAT A HYDROGEN SULPHIDE COMPRISING GAS

(71) Applicant: Paques I.P. B.V., Balk (NL)

(72) Inventors: Johannes Bernardus Maria KLOK, El Balk (NL); Dhaval MEHTA, El Balk (NL); Hendrik DIJKMAN, El Balk (NL); Annemerel Rozemarijn MOL, El Balk (NL); Renata Dorothea VAN DER WEIJDEN, El Balk (NL); Cees Jan Nico BUISMAN, El Balk (NL)

(21) Appl. No.: 18/390,290

(22) Filed: Dec. 20, 2023

Related U.S. Application Data
(63) Continuation of application No. PCT/NL2022/050345, filed on Jun. 20, 2022.

(30) Foreign Application Priority Data
Jun. 21, 2021 (NL) 2028503

(51) Int. Cl. B01D 53/84; B01D 53/14; B01D 53/18; B01D 53/52; B01D 53/78; B01D 53/96; C01B 17/02; C02F 3/34; C02F 101/10; C02F 103/18; C12P 3/00

(52) U.S. Cl. CPC B01D 53/84; B01D 53/1425; B01D 53/1462; B01D 53/18; B01D 53/526; B01D 53/78; B01D 53/96; C01B 17/0205; C02F 3/345; C12P 3/00; B01D 2251/304; B01D 2251/306; B01D 2251/606; C02F 2101/101; C02F 2103/18

(57) ABSTRACT

The invention is directed to a process to continuously treat a hydrogen sulphide comprising gas, said process comprising the following steps:
(a) contacting the hydrogen sulphide comprising gas with an aqueous alkaline liquid comprising sulphide-oxidising bacteria and elemental sulphur particles thereby producing a loaded aqueous liquid comprising dissolved sulphide, polysulphide compounds, sulphide-oxidising bacteria and elemental sulphur particles and a gas having a lower content of hydrogen sulphide, and passing the loaded aqueous liquid through a polysulphide reactor zone comprising one or more plug flow reactor zones,
(b) contacting the loaded aqueous liquid with an oxidant to enable the sulphide-oxidising bacteria to oxidise sulphide to elemental sulphur, thereby producing an enriched aqueous liquid comprising an increased amount of elemental sulphur particles and
(c) separating elemental sulphur particles from the enriched aqueous liquid, wherein the residence time of the loaded aqueous liquid between its preparation in step (a) and its supply to step (b) is between 3 and 45 minutes, and wherein the content of elemental sulphur as part of the polysulphide compounds in the loaded aqueous liquid [S⁰ in Sₓ²⁻] as supplied to step (b) is above 0.7 mM.

This application is a continuation of International Patent Application No. PCT/NL2022/050345 filed Jun. 20, 2022, which application claims priority to Netherlands Patent Application No. 2028503 filed Jun. 21, 2021, the contents of which are both incorporated herein by reference in their entireties.

The invention is directed to a process to continuously treat a hydrogen sulphide comprising gas, said process comprising the following steps: (a) contacting the hydrogen sulphide comprising gas with an aqueous alkaline liquid further comprising sulphide-oxidising bacteria and elemental sulphur particles, thereby producing a loaded aqueous liquid, (b) contacting the loaded aqueous liquid with an oxidant wherein sulphide is oxidised to elemental sulphur by the sulphide-oxidising bacteria, thereby producing an enriched aqueous liquid comprising an increased amount of elemental sulphur particles and (c) separating elemental sulphur particles from the enriched aqueous liquid. The invention is also directed to a sulphur reclaiming process facility.

Such a biological desulfurization process is described in WO92/10270. The process as described in this publication has been applied in already more than 250 commercial installations worldwide. Sulphur particle separation, however, remains a challenge; a fraction of the sulphur particles is often too small for liquid-solid separation with conventional separation technology.

Sulfur is the 10th most abundant element in the universe and plays a vital role in the Earth's ecosystem through the (bio)chemical sulfur cycle. It can be present in various oxidation states, from hydrogen sulphide (H₂S) being the most reduced state (−2) to sulfate (SO₄²⁻) the most oxidized state (+6). Elemental sulfur (S with an oxidation state of zero), can be recovered from hydrogen sulphide comprising gas using the process as described in WO92/10270. Main advantages of this process in comparison to chemical and physical alternatives are operation at ambient pressure and temperature and without toxic chemicals. In the process of WO92/10270 H₂S as is absorbed in a moderately alkaline solution where it reacts to soluble bisulphide and is subsequently oxidized by a mixed culture of sulphide-oxidizing bacteria to elemental sulfur. The elemental sulphur is predominantly present in the form of orthorhombic α-S₈. Next to formation of elemental sulfur, oxidized by-products such as sulfate and thiosulfate are formed due to overexposure to dissolved oxygen wherein sulfate is formed biologically and thiosulfate is formed abiotically. These compounds are undesirable as they lead to acidification and consequently addition of chemicals is needed to neutralize the process solution.

Although the above described biological desulfurization process has been intensively studied the sulphur settleability is still a major challenge. Sulphur is found to settle poorly and the settleability of the produced sulphur in commercial processes fluctuates over time. Poorly settleable sulphur may lead to hampered process operation, as it accumulates in the system. Accumulated sulphur can cause problems such as clogging of pumps and pipes, and under high concentrations to foaming as well. Moreover, small sulphur particles are more prone to side reactions such as oxidation, due to their larger relative surface area.

The object of this invention is to provide a process and sulphur reclaiming facility which does not have the afore mentioned problems regarding poor and fluctuating sulphur settleability.

This is provided with the following process. A process to continuously treat a hydrogen sulphide comprising gas, said process comprising the following steps:
(a) contacting the hydrogen sulphide comprising gas with an aqueous alkaline liquid further comprising sulphide-oxidising bacteria and elemental sulphur particles, thereby producing a loaded aqueous liquid comprising dissolved sulphide, polysulphide compounds, sulphide-oxidising bacteria and elemental sulphur particles and a gas having a lower content of hydrogen sulphide, and passing the loaded aqueous liquid through a polysulphide reactor zone comprising one or more plug flow reactor zones,
(b) contacting the loaded aqueous liquid with an oxidant to enable the sulphide-oxidising bacteria to oxidise sulphide to elemental sulphur, thereby producing an enriched aqueous liquid comprising an increased amount of elemental sulphur particles, and
(c) separating elemental sulphur particles from the enriched aqueous liquid, wherein the residence time of the loaded aqueous liquid between its preparation in step (a) and its supply to step (b) is between 3 and 45 minutes, and wherein the content of the elemental sulphur as part of the polysulphide compounds in the loaded aqueous liquid [S⁰ in Sₓ²⁻] as supplied to step (b) is above 0.7 mM.

Applicants have found that the settleability of elemental sulphur is significantly improved when the process is performed as claimed. Results show that agglomeration of sulphur particles is promoted while the presence of very small single particles is lower compared to the prior art process conditions. It is surprising that a high content of polysulphide compounds in the loaded aqueous liquid is beneficial because polysulphide formation in the bioreactor of step (b) was deemed unwanted, as it indicates a poor operating condition. Furthermore, polysulphides are known to be more sensitive for over oxidation than sulphide, which results in a lower efficiency of elemental sulphur formation/regeneration of caustic which is unwanted. Applicant believes that the improvement regarding settlement of elemental sulphur results from the fact that very small sulphur particles are removed in step (a) and before performing step (b) as a result of the presence of polysulphides. Due to polysulphide formation the remaining smallest particles in the liquid are believed to have a higher tendency to agglomerate resulting in an improved settlement of elemental sulphur.

Without wishing to be bound by the following theory applicant believes that the improved settlement of elemental sulphur can be explained by the following equilibrium between polysulphide and S8 rings:

$${HS}^{-} + ( x - 1 ) / 8 {S}_{8} \text{<-} -> {S}_{x}^{2 -} + {H}^{+} \qquad ( 1 )$$

When the content of polysulphide, [Sₓ²⁻], is high the formation of dissolved S8 rings will also be promoted resulting in a so-called super saturation of dissolved S8 rings which in turn results in the desired formation of aggregates under so-called polysulphidic conditions. These polysulphidic conditions are expressed by the following formula (2) wherein [S⁰ in Sₓ²⁻] is the content of elemental sulphur as part of the polysulphide compounds [Sₓ²⁻] in the loaded aqueous liquid as supplied to step (b):

$$[ {S}^{0} in {S}_{x}^{2 -} ] \geq 1.7 * [ {HS}^{-} ] * {10}^{( - 9.17 + pH )} \qquad ( 2 )$$

wherein [S⁰ in Sₓ²⁻] is the content of elemental sulphur as part of the polysulphide compounds in the loaded aqueous liquid as supplied to step (b) expressed in mM S, [HS⁻] is the sulphide concentration expressed in mM of the loaded aqueous liquid as supplied to step (b), and pH is the pH of the loaded aqueous liquid as supplied to step (b).

Even more preferably, the polysulphidic conditions are expressed by the following formula (3)

$$[ {S}^{0} in {S}_{x}^{2 -} ] \geq 2.8 ⋆ [ {HS}^{-} ] * {10}^{( - 9.17 + pH )} \qquad ( 3 )$$

Typically,

$$[ {S}^{0} in {S}_{x}^{2 -} ] \leq 6. ⋆ [ {HS}^{-} ] * {10}^{( - 9.17 + pH )} \qquad ( 4 )$$

The content of the elemental sulphur as part of the polysulphide compounds in the loaded aqueous liquid [S⁰ in Sₓ²⁻] as supplied to step (b) is above 0.7 mM and preferably above 1 mM and even more preferably above 1.5 mM.

The content of the elemental sulphur as part of the polysulphide compounds in the loaded aqueous liquid [S⁰ in Sₓ²⁻] depends on the content of polysulphide, [Sₓ²⁻] and the average chain length x according to the following formula:

$$[ {S}^{0} in {S}_{x}^{2 -} ] = ( x - 1 ) * [ {S}_{x}^{2 -} ] \qquad ( 3 )$$

For example, for a polysulphide wherein x=4 according to the following formula S—S—S—S²— and present in a concentration [Sₓ²⁻] of 1.5 mM the content of elemental sulphur as part of the polysulphide compounds [S⁰ in Sₓ²⁻] is:

$$( 4 - 1 ) \times 1.5 mM = 4.5 mM .$$

Polysulphides are formed by reaction between bisulphide and elemental sulphur. The polysulphide itself may also react with elemental sulphur. It is believed that due to the high surface to volume ratio of the very small elemental sulphur particles these particles are selectively consumed. This chemical reaction obviously also takes place in the prior art processes. However in the prior art processes the concentration of polysulphides does not reach the level that is achieved in the present process and that results in the improved sulphur settlement. For this reason it is preferred to operate the process according to this invention for a period of at least 1 week such that the daily average content of elemental sulphur as part of the polysulphide compounds in the loaded aqueous liquid [S⁰ in Sₓ²⁻] as supplied to step (b) is above 0.7 mM, preferably above 1 mM and even more preferably above 1.5 mM. More preferably to operate the process according to this invention for a period of at least 1 week under the polysulphidic conditions as expressed by the above formula (2).

The hydrogen sulphide comprising gas may be any gas comprising such a compound. The hydrogen sulphide comprising gas may also comprise carbon dioxide, nitrogen, hydrogen, small amounts of oxygen, water vapour and gaseous hydrocarbons, such as for example methane, ethane, propane and/or higher boiling hydrocarbons, and mercaptans and/or other sulphur compounds, for example carbonyl sulphide. Such a gas may be natural gas, bio-gas from for example anaerobic wastewater treatment units, refinery off gas, synthesis gas, geothermal gas, landfill gas or acid gas obtained in an amine gas treating process. The invention is especially suited for gasses having a content of carbon dioxide of above 20 vol % and a hydrogen sulphide content of between 0.1 and 3 vol. %. When such gasses are treated by the prior art processes sulphur settleability is especially a challenge. With hindsight it is believed that a low content of sulphur as part of polysulphide at the resulting lower pH and the lower bisulphide content cause a poor settlement of elemental sulphur as also illustrated in Example B. When such gasses are treated by the process of this invention an improved settleability of elemental sulphur is observed.

In step (a) the hydrogen sulphide comprising gas is contacted with an aqueous alkaline liquid further comprising sulphide-oxidising bacteria and at a temperature of preferably between 15 and 48° C. and more preferably between 35 and 45° C. Such a process is also referred to as an absorption process and is typically performed in an absorption or contacting column where gas and liquid flow counter-currently.

Suitably step (a) is performed in a vertical column wherein continuously the hydrogen sulphide comprising gas is fed to the column at a lower position of the column and the aqueous liquid comprising sulphide-oxidising bacteria is continuously fed to a higher position of the column such that a substantially upward flowing gaseous stream contacts a substantially downwards flowing aqueous stream. The column is further provided with an outlet for the loaded aqueous liquid at its lower end and an outlet for treated gas at its upper end.

The aqueous alkaline liquid may be any liquid alkaline absorbent known to be suitable for absorption of hydrogen sulphide. Examples of suitable liquid alkaline absorbents are carbonate, bicarbonate and/or phosphate solutions and more preferably the aqueous liquid is a buffered liquid further comprising sodium carbonate and sodium bicarbonate or potassium carbonate and potassium bicarbonate or their mixtures. The pH of the liquid aqueous alkaline liquid is preferably in the range of from 7 to 10, more preferably of from 7.5 to 9.5. It will be appreciated that in downward direction of the column, the pH of the absorption liquid will decrease due to absorption of hydrogen sulphide and carbon dioxide. The pH of the loaded aqueous liquid produced in step (a) will be typically lower than the pH of the aqueous liquid provided to the absorption column. The pH of the loaded aqueous liquid produced in step (a) may be as low as 6.5 and is preferably in the range of from 6.5 to 9.0.

The pressure in step (a) may be up to 100 bara, preferably of from atmospheric pressure to 80 bara.

The concentration of oxygen and especially dissolved oxygen is low in step (a). The concentration of molecular oxygen in the loaded aqueous liquid produced in step (a) is at most 10 μM, preferably at most 1 μM, more preferably at most 0.1 μM. To achieve these low oxygen contents the oxygen content in the hydrogen sulphide comprising gas is suitably below 3 vol. % and preferably below 1 vol. %.

The sulphide-oxidising bacteria as present in step (a) may be any sulphide-oxidising bacteria, preferably sulphide-oxidising bacteria of the genera Halothiobacillus, Thioalkalimicrobium, Thioalkalispira, Thioalkalibacter, Thioalkalivibrio, Alkalilimnicola and related bacteria. The sulphide-oxidising bacteria as present in step (a) may be provided by recirculating the enriched aqueous liquid from step (b) and/or by recirculating aqueous liquid obtained after removal of elemental sulphur particles in step (c). It has been found that when such sulphide-oxidising bacteria are present in the aqueous alkaline liquid under the above described conditions a very effective absorption of hydrogen sulphide results. The content of sulphide-oxidising bacteria, based on nitrogen content, in the aqueous alkaline liquid in step (a) is preferably greater than 5 mg N/L and lower than 1000 mg N/L and more preferably between 25 and 200 mg N/L.

The loaded aqueous liquid as produced in step (a) comprises dissolved bisulphide, elemental sulphur particles and sulphide oxidising bacteria. The combined concentration of bisulphide, polysulphide compounds, sulphur in sulphide-oxidising bacteria and elemental sulphur in the loaded aqueous liquid produced in step (a) (expressed as sulphur) may be up to 20 grams per litre. Preferably this combined concentration in the loaded aqueous liquid is in the range of from 100 mg/L to 15 g/L, more preferably of from 150 mg/L to 10 g/L. The aqueous liquid may comprise trace compounds, such as for example iron, copper or zinc, as nutrients for the sulphide-oxidising bacteria.

The elemental sulphur particles in the aqueous alkaline liquid of step (a) may suitably be provided by recirculating at least a part of the enriched aqueous liquid of step (b) to step (a). In a particularly preferred embodiment, the bulk of the aqueous alkaline liquid that is provided in step (a) consists of recirculated enriched aqueous liquid from step (b). More preferably, at least 90 vol. % of the alkaline liquid that is provided in step (a) consists of recirculated enriched aqueous liquid from step (b).

In step (b) the loaded aqueous liquid of step (a) is contacted with an oxidant wherein sulphide is oxidised to elemental sulphur by the sulphide-oxidising bacteria. Preferably, the amount of oxidant supplied to the bioreactor in which step (b) may be performed is at least about the stoichiometric amount needed for oxidation of the sulphide in step (a) and/or (b) into elemental sulphur. In this way the protons generated by the bacteria when forming elemental sulphur are consumed thereby regenerating the bacteria. This step is therefore also referred to as a caustic regeneration. The thus obtained regenerated sulphide-oxidising bacteria may subsequently be reused in step (a). Any suitable oxidant may be used, for example nitrate or molecular oxygen, preferably molecular oxygen. The oxidant may be supplied in any suitable way, preferably by supplying a gaseous stream comprising molecular oxygen to a bioreactor. The gaseous stream comprising molecular oxygen may be any suitable gas comprising oxygen, preferably air.

Preferably the temperature in step (b) is in the range of from 10 to 48° C., more preferably of from 35 to 45° C. and the pressure is between 0 bara and 10 bara, more preferably from atmospheric pressure to 5 bara, even more preferably at atmospheric pressure.

The elemental sulphur particles in the enriched aqueous liquid that is formed in step (b) is separated in step (c). Such a separation may be performed after step (b) has been completed and/or it may be performed simultaneously with step (b). This isolation of elemental sulphur may be performed by any means known in the art, such as for example by means of sedimentation or other means for solid-liquid separation. Preferably elemental sulphur is recovered by taking part of the aqueous solution obtained in step (b) and isolating elemental sulphur from that part to obtain a sulphur-depleted effluent. Part of the sulphur depleted effluent may be recycled to step (b) and part of the sulphur depleted effluent may be purged. Another part of the aqueous solution obtained in step (b) may be used as the aqueous alkaline solution in step (a).

The content of elemental sulphur as part of the polysulphide compounds in the loaded aqueous liquid [S⁰ in Sₓ²⁻] is measured according to the following method. The total concentration of polysulphide is first measured spectrophotometrically at a wavelength of 285 nm as described by Kleinjan, W. E.; De Keizer, A.; Janssen, A. J. H., Equilibrium of the reaction between dissolved sodium sulphide and biologically produced sulphur. Colloids and Surfaces B: Biointerfaces 2005, 43, (3-4), 228-237. The average chain length is subsequently determined by thermodynamics as described by Alexey Kamyshny, Jenny Gun, Dan Rizkov, Tamara Voitsekovski, and Ovadia Lev in Environ. Sci. Technol. 2007, 41, 7, 2395-2400. The content of elemental sulphur as part of the polysulphide compounds [S⁰ in Sₓ²⁻] can now be calculated making use of the measured polysulphide content and the determined average chain length.

The required content of elemental sulphur as part of the polysulphide compounds in the loaded aqueous liquid [S⁰ in Sₓ²⁻] or the polysulphidic conditions may be achieved in the process by influencing temperature, residence time and pH or a combination of these measures. Higher sulphide concentration, higher temperature, longer residence time and higher pH favour the formation of polysulphides. Also local high contents of polysulphide and small elemental sulphur particles may further result in a higher polysulphide content at a similar reaction time due to autocatalytic effects. Thus the skilled person may choose various measures to achieve the process conditions of the present invention to perform the process having a good and stable sulphur settlement.

One way of influencing the temperature is wherein the aqueous alkaline liquid is increased in temperature by indirect heat exchange with the loaded aqueous liquid and/or an external heat source thereby obtaining a heated aqueous alkaline liquid which is used in step (a). The loaded aqueous liquid, for example just before it is used in step (b), will suitably have a higher temperature than the loaded aqueous liquid as supplied to step (a). Suitably the loaded aqueous liquid may have a temperature of between 35 and 50° C. By using this relatively warm stream to increase the temperature of the aqueous alkaline liquid which is to be used in step (a) the required content of elemental sulphur as part of the polysulphide compounds in the loaded aqueous liquid [S⁰ in Sₓ²⁻] or the polysulphidic conditions according to this invention may be achieved.

In order to achieve a high content of elemental sulphur as part of the polysulphide compounds in the loaded aqueous liquid as supplied to step (b), the loaded aqueous liquid needs to be given sufficient residence time. By increasing residence time of the loaded aqueous liquid between its preparation in step (a) and its supply to step (b) the formation of elemental sulphur as part of the polysulphide compounds in the loaded aqueous liquid [S⁰ in Sₓ²⁻] or the polysulphidic conditions is promoted. This residence time is also referred to as the Sulphidic′ Retention Time (SuRT). Preferably, the residence time SuRT is between 3 and 45 minutes, more preferably between 5 and 15 minutes.

The desired residence time is achieved by a process wherein step (a) comprises passing the loaded aqueous liquid flows through a polysulphide reactor zone. In this polysulphide reactor zone polysulphide compounds are formed by reaction of the dissolved sulphide and the elemental sulphur particles. The polysulphide reactor zone comprises one or more plug flow reactor zones. In these plug flow reactor zones back mixing is minimised. The polysulphide reactor zone will as a result have a different content of polysulphide in different regions of the polysulphide reactor zone. This is beneficial because as a result of the autocatalytic nature of the reaction a higher average polysulphide concentration and a higher polysulphide concentration in the effluent of the polysulphide reactor zone is achieved. The term “plug flow reactor zone” as used herein refers to a zone within a tube through which a fluid is flowing, in which zone the velocity of the fluid is substantially constant across any cross-section of the tube perpendicular to the axis of the pipe, assuming that there is no boundary layer adjacent to the inner wall of the tube.

The polysulphide reactor zone may for example be a vertically or horizontally extended vessel having internals along which the liquid flows in a zig-zag flow pattern through the vessel. Such a polysulphide reactor zone will thus have an upstream region and a down stream region. Further the loaded aqueous liquid will have a higher polysulphide content in the downstream region compared to the upstream region.

The required content of elemental sulphur as part of the polysulphide compounds in the loaded aqueous liquid [S⁰ in Sₓ²⁻] or the polysulphidic conditions may be achieved in the polysulphide reactor zone by recycling part of the loaded aqueous liquid having a higher polysulphide content from a downstream region of the polysulphide reactor zone to an upstream region in the polysulphide reactor zone where the loaded aqueous liquid has a lower polysulphide content with the object to increase the polysulphide content in the upstream region. It has been found that the presence of polysulphide compounds enhances the formation rate of more polysulphide compounds. Thus by recycling in this way this autocatalytic effect is enhanced resulting in that the conditions of this invention are even more achieved. Suitably between 5 and 50 wt % of the loaded aqueous liquid as discharged at the downstream region is recycled to the upstream region.

Preferably the part of the loaded aqueous liquid having a higher polysulphide content which is recycled is not immediately added to the upstream region in the polysulphide reactor zone, like by means of a conduit and pump. By not immediately adding this part extra residence time for this part will be created thereby allowing that even more polysulphide will form in this part. By adding this polysulphide enriched part to the upstream region in the polysulphide reactor zone an even greater autocatalytic effect will be present. Suitably the part of the loaded aqueous liquid as isolated from the downstream region flows via a zone having a residence time of between 5 and 45 minutes before it is recycled to the upstream region in the polysulphide reactor zone. Preferably the residence time in this zone is between 5 and 15 minutes.

The required content of elemental sulphur as part of the polysulphide compounds in the loaded aqueous liquid [S⁰ in Sₓ²⁻] or the polysulphidic conditions may be achieved by directly supplying part of the aqueous alkaline liquid further comprising sulphide-oxidising bacteria to the polysulphide reactor zone. This part thus by-passes the absorption, i.e. the contacting part of step (a) and is directly mixed with the loaded aqueous liquid in the polysulphide reactor zone. Preferably part of the aqueous alkaline liquid is added to the upstream region of the polysulphide reactor zone. Suitably between 5 and 20 wt % of the aqueous alkaline liquid is directly provided to the polysulphide reactor zone while the remaining part is used to contact with the hydrogen sulphide comprising gas.

Increasing the polysulphide content in the part of the loaded aqueous liquid which is recycled to the upstream region in the polysulphide reactor zone may also be achieved by increasing the temperature of this part. Preferably part of the loaded aqueous liquid having a higher polysulphide content is increased in temperature before being recycled to the upstream region in the polysulphide reactor zone. Preferably the temperature is increased to a temperature between 35 and 50° C.

As also described above, step (a) is preferably performed in a vertical column wherein continuously the hydrogen sulphide comprising gas is fed to the column at a lower position of the column and the aqueous liquid comprising sulphide-oxidising bacteria is continuously fed to a higher position of the column such that a substantially upward flowing gaseous stream contacts a substantially downwards flowing aqueous stream. Preferably part of the aqueous liquid comprising sulphide-oxidising bacteria is continuously fed to a higher position of the column to contact with the upflowing gaseous stream in a first contacting zone which generates an intermediate loaded aqueous liquid. In the first contacting zone the gas is polished to its required low level of hydrogen sulphide. Part of the aqueous liquid comprising sulphide-oxidising bacteria is continuously fed to an intermediate position of the column to contact together with the intermediate loaded aqueous liquid, with the upflowing gaseous stream in a second contacting zone. In this second contacting zone part of the aqueous liquid comprising sulphide-oxidising bacteria contacts fresh feed gas resulting in a higher polysulphide concentration in the combined loaded aqueous liquid. By mixing this loaded aqueous liquid with the intermediate loaded aqueous liquid any small sulphur particles present in the intermediate loaded aqueous liquid will be reacted away by the polysulphides. Preferably between 5 to 50 wt % of the total aqueous liquid comprising sulphide-oxidising bacteria is supplied to this second contacting zone. This embodiment may be performed in a single absorption vessel.

In step (a), in another preferred embodiment, part of the aqueous liquid comprising sulphide-oxidising bacteria is continuously contacted in a step (a1) with the hydrogen sulphide comprising gas to obtain a first intermediate loaded aqueous liquid and an intermediate gas having a lower intermediate content of hydrogen sulphide and another part of the aqueous liquid comprising sulphide-oxidising bacteria is continuously contacted in a step (a2) with the intermediate gas having a lower intermediate content of hydrogen sulphide to obtain a second intermediate loaded aqueous liquid and the gas having a lower content of hydrogen sulphide. Step (a2) may be considered to be a polishing step where the required low level of hydrogen sulphide is achieved. In step (a1) part of the aqueous liquid comprising sulphide-oxidising bacteria contacts fresh feed gas resulting in a higher polysulphide concentration in the first intermediate loaded aqueous liquid. By combining the first intermediate loaded aqueous liquid with the second intermediate loaded aqueous liquid any small sulphur particles present in the second intermediate loaded aqueous liquid will be reacted away by the polysulphides. Preferably between 5 to 50 wt % of the total aqueous liquid comprising sulphide-oxidising bacteria is supplied to step (a1). The first intermediate loaded aqueous liquid is combined with the second intermediate loaded aqueous liquid to obtain the loaded aqueous liquid. The residence time of the first and second intermediate loaded aqueous liquids between step (a) and step (b) is suitably between 5 and 45 minutes and preferably between 5 and 15 minutes.

Preferably each first and second intermediate loaded aqueous liquids produced in step (a1) and (a2) flow through separate first and second polysulphide reactor zones respectively. In the polysulphide reactor zones polysulphide compounds are formed by reaction of the dissolved sulphide and the elemental sulphur. Preferably the polysulphide reactor zones comprise one or more plug flow reactor zones which avoid back mixing as described above. Preferably part of the first intermediate loaded aqueous liquid rich in polysulphides is supplied to the second polysulphide reactor zone to increase the polysulphide content in the second intermediate loaded aqueous liquid. When the polysulphide reactor zones have an upstream and downstream region it is preferred that the part of the first intermediate loaded aqueous liquid rich in polysulphides is supplied to the upstream region of the second polysulphide reactor zone to increase the polysulphide content in the second intermediate loaded aqueous liquid.

Step (a1) and step (a2) may be performed in the same or preferably separate absorption columns. More preferably step (a1) and step (a2) are performed in separate absorption columns, each comprising a lower end where the respective polysulphide reactor zones are present.

The process is suitably performed by making use of a measurement and control where the content of elemental sulphur as part of the polysulphide compounds in the loaded aqueous liquid [S⁰ in Sₓ²⁻] or the polysulphidic conditions in the loaded aqueous liquid as supplied to step (b) is measured and when the measured content is below a threshold value the temperature and/or residence time by influencing the recycle is adapted with the object to increase said content as described above.

The invention will be illustrated by means of FIGS. 1-7.

### BRIEF DESCRIPTION OF THE FIGURES

FIG. 1 shows a lab-scale reactor set-up.

FIG. 2 shows typical particle size distributions (PSD) of sulfur particle samples taken from the microaerophilic reactor during Experiment A and Examples 1-3.

FIGS. 3a-3d show the distinctively different morphologies of sulfur particles formed under the various experimental conditions as observed with light microscopy in Comparative Experiment A (FIG. 3a), Example 1 (FIG. 3b), Example 2 (FIG. 3c) and Example 3 (FIG. 3d).

FIG. 4 shows modelling results support the experimentally obtained findings that the smallest sulfur particles dissolve in the polysulphide reactor zone, due to polysulphide formation to the extent that the conditions allowed for, including equilibrium Sₓ²⁻ between sulfur, sulphide and polysulphide was reached for Examples 1, 2 and 3.

FIG. 5 shows a sulphur reclaiming process facility in which the process according to the present invention may be performed.

FIG. 6 shows another sulphur reclaiming process facility in which the process according to the present invention may be performed.

FIG. 7 shows yet another sulphur a reclaiming process.

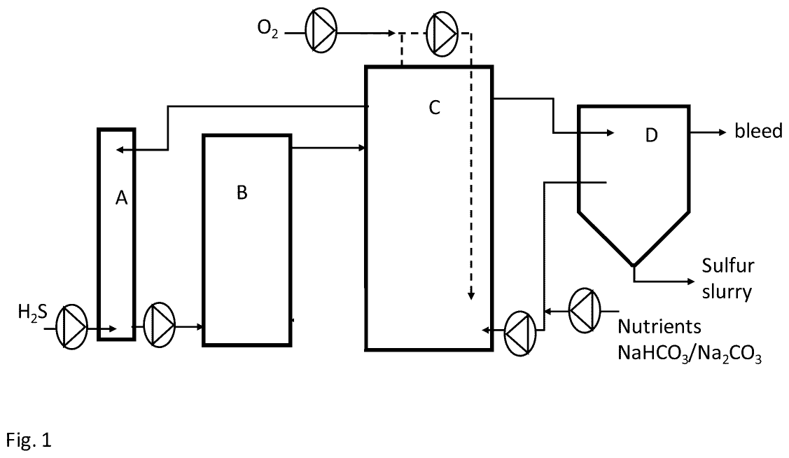

FIGS. 1-4 relate to the Examples.

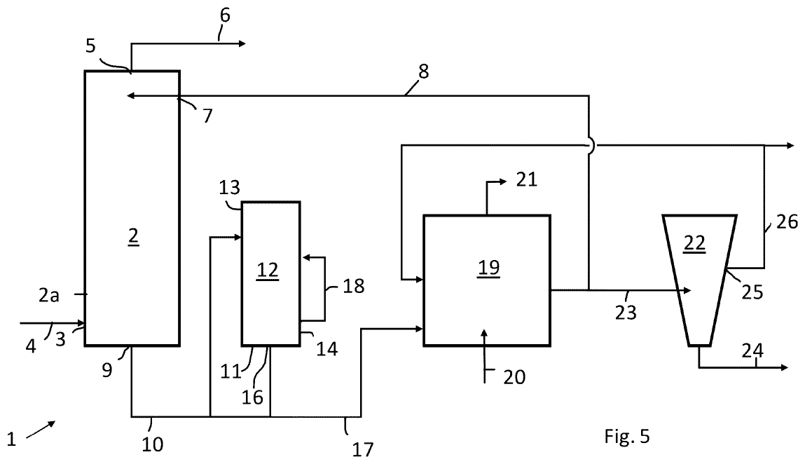

FIG. 5 shows a sulphur reclaiming process facility in which the process according to the present invention may be performed. The invention is also directed to this sulphur reclaiming process facility. The sulphur reclaiming process facility (1) is provided with an absorption column (2) provided with an inlet (3) for a hydrogen sulphide comprising gas (4), an outlet (5) for a gas (6) having a lower content of hydrogen sulphide at its upper end, an inlet (7) for an aqueous alkaline liquid (8) further comprising sulphide-oxidising bacteria and a first outlet (9) for a loaded aqueous liquid (10) at a lower elevation. Further a polysulphide reactor zone (11) is shown. This polysulphide reactor zone (11) is part of a separate vessel (12). Alternatively the polysulphide reactor zone (11) may also be positioned in the lower end (2a) of the absorption column (2) or be a combination of these two embodiments. The polysulphide reactor zone (11) comprises a plug flow reactor zone, said polysulphide reactor zone (11) comprising an upstream end (13) and a downstream end (14). The upstream end (13) of the polysulphide reactor zone (11) is fluidly connected to the first outlet (9) for a loaded aqueous liquid (10). The downstream end (14) of the polysulphide reactor zone (11) is provided with a second outlet (16) for the loaded aqueous liquid (17) and with a recycle stream (18) for part of the loaded aqueous liquid to the upstream end (13) of the polysulphide reactor zone (11). This recycle stream (18) achieves that the content of elemental sulphur as part of polysulphide is increased in the loaded aqueous liquid as it is supplied to an aerobic operated bioreactor (19). This content may be increased or decreased by increasing or decreasing the fraction which is recycled. The content of elemental sulphur as part of polysulphide may also be increased by increasing the temperature of the recycle stream (18), by increasing the temperature in the lower end (2a) of the absorption column (2), by increasing the temperature in separate vessel (12) and/or by increasing the time between isolating part of the loaded aqueous fraction from the downstream region (14) and supplying this part at the upstream region (13).

The second outlet (16) for the loaded aqueous liquid (17) is fluidly connected to an aerobic operated bioreactor (19) for performing step (c) of the process. To the aerobic operated bioreactor (19) air (20) is provided and used air (21) is discharged. The aerobic operated bioreactor (19) is fluidly connected to the inlet (7) for the aqueous alkaline liquid (8) of the absorber column (2) and to an elemental sulphur recovery unit (22) via conduit (23). The recovery unit (22) may alternatively be part of bioreactor (19). The elemental sulphur recovery unit (22) is provided with an outlet (24) for elemental sulphur and an outlet (25) for a liquid effluent (26) poor in elemental sulphur. This liquid effluent is partly purged and partly returned to the aerobic operated bioreactor (19) as shown.

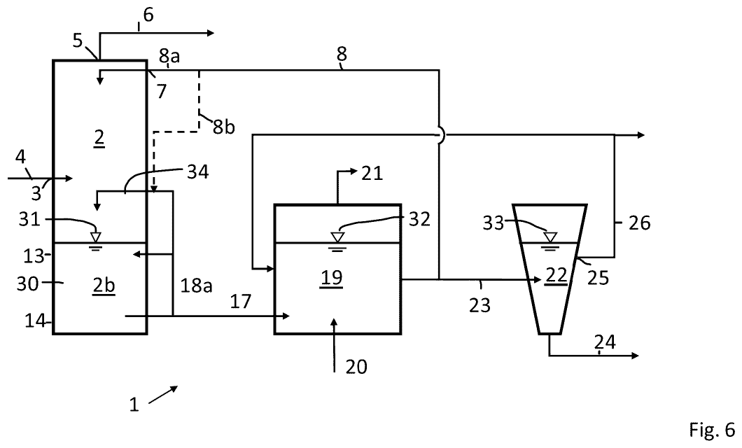

FIG. 6 shows a sulphur reclaiming process facility as in FIG. 5 with the following differences. Instead of a separate vessel (12) the polysulphide reactor zone (11) is located in the bottom part (2b) of the absorption column (2) as a so-called sump (30). The sump (30) is a volume of loaded aqueous liquid (17) defined at its upper end by a liquid level (31). This liquid level (31) may be at the same elevation as the liquid levels (32) in the aerobic operated bioreactor (19) and the liquid level (33) in the elemental sulphur recovery unit (22) as shown or may be at different elevations. The inlet (3) for a hydrogen sulphide comprising gas (4) is located above the liquid level (31) of sump (30). The sump (30) as the polysulphide reactor zone (11) comprises one or more plug flow reactor zones, said polysulphide reactor zone comprising an upstream end (13) and a downstream end. Part of the loaded aqueous liquid (17) is recycled via recycle stream (18a) to the upstream region (13) of the sump (30). As shown this part may also be supplied to the absorption column (2) at a position (34) above liquid level (31) of sump (30) as recycle stream (18a). This recycle stream (18a) may be combined with part (8b) of the aqueous alkaline liquid (8). Another part (8a) of aqueous alkaline liquid (8) is provided to inlet (7) at the upper end of column (2).

This recycle stream (18a) achieves that the content of elemental sulphur as part of polysulphide is increased in the loaded aqueous liquid (17) before it is supplied to an aerobic operated bioreactor (19). This content may be increased or decreased by increasing or decreasing the fraction (18a) which is recycled. The content of elemental sulphur as part of polysulphide may also be increased by increasing the temperature of the recycle stream (18a), by increasing the temperature in sump (30) and/or by increasing the time between isolating part of the loaded aqueous fraction from the downstream region (14) and supplying this part at the upstream region (13) or to position (34).

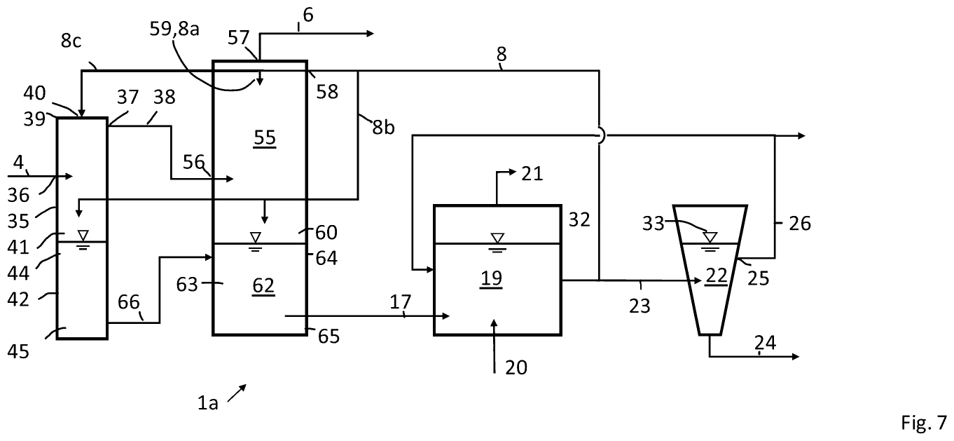

FIG. 7 shows a sulphur a reclaiming process facility (1a) comprising a first absorption column (35) provided with an inlet (36) for a hydrogen sulphide comprising gas (4), an outlet (37) for an intermediate gas (38) having a lower content of hydrogen sulphide at its upper end (39), an inlet (40) for part (8c) of an aqueous alkaline liquid (8) further comprising sulphide-oxidising bacteria and an outlet (41) for a first intermediate loaded aqueous liquid at a lower elevation. Also a second absorption column (55) is shown provided with an inlet (56) for the intermediate gas (38) having a lower content of hydrogen sulphide, an outlet (57) for a gas (6) having a lower content of hydrogen sulphide at its upper end (58), an inlet (59) for part (8a) of an aqueous alkaline liquid (8) further comprising sulphide-oxidising bacteria and a outlet (60) for a second intermediate loaded aqueous liquid at a lower elevation.

A polysulphide reactor zone (42) is part of the first absorption column (35) and positioned in the lower end (43) of the first absorption column (35). A polysulphide reactor zone (62) is part of the second absorption column (55) and positioned in the lower end (63) of the second absorption column (55). The polysulphide reactor zones (42,62) comprise one or more plug flow reactor zones, said sulphide reactor zones (42,62) comprising an upstream end (44,64) and a down stream end (45,65).

The upstream end (44) of the polysulphide reactor zone (42) of the first absorption column (35) is fluidly connected to the outlet (41) for the first intermediate loaded aqueous liquid and the upstream end (64) of the polysulphide reactor zone (62) of the second absorption column (55) is fluidly connected to the outlet (60) for the second intermediate loaded aqueous liquid. The downstream end (45) of the polysulphide reactor zone (42) of the first absorption column (35) is fluidly connected to the upstream end (64) of the polysulphide reactor zone (62) of the second absorption column (55) via stream (66). In this manner a fraction comprising high contents of polysulphide are added to the polysulphide reactor zone (62). The resulting loaded aqueous liquid (17) will then have the claimed properties. This loaded aqueous liquid (17) is supplied to an aerobic operated bioreactor (19) for oxidation of sulphide to elemental sulphur. For this the downstream end (65) of the polysulphide reactor zone (62) of the second absorption column (55) is fluidly connected to an aerobic operated bioreactor (19) for regeneration of the sulphide-oxidising bacteria.

Part of the contents of the polysulphide reactor zone (42) of the first absorption column (35) may be directly supplied to bioreactor (19) (not shown). Part (8b) of the aqueous alkaline liquid (8) further comprising sulphide-oxidising bacteria is added to first and second absorber columns (35,55) to further enhance the formation of polysulphides. The first absorber column may have a simple design, not necessarily provided with contacting internals. Step (a1) described above may be performed in the first absorber column (35). Second absorber column (55) is suitably provided with contacting internals such to optimise the gas-liquid contacting such to achieve an optimal absorption of the hydrogen sulphide. Step (a2) described above may be performed in the second absorber column (55).

The aerobic operated bioreactor (19) is fluidly connected to the inlet (59) for a part (8a) of the aqueous alkaline liquid (8) of the second absorber column and fluidly connected to the inlet (40) for a part (8c) of the aqueous alkaline liquid (8) of the first absorber column. The elemental sulphur recovery unit (22) is provided with an inlet fluidly connected to the aerobic operated bioreactor (19) and provided with an outlet (24) for elemental sulphur and an outlet (25) for a liquid effluent poor in elemental sulphur.

The polysulphide reactor zone (11,2b, 42,62) of FIGS. 5-7 may be provided with means to increase the temperature of the liquid contents of the polysulphide reactor zone (11,2a, 42,62). This may be by indirect heat exchange wherein for example a hot heating medium flows through tubes as present in the polysulphide reactor zone (11,2b, 42,62) thereby heating up the liquid contents of these zones.

The invention will be illustrated by the following non-limiting experiments.

### EXAMPLES

Here we report the effects of a novel sulphidic reactor inserted in the conventional process set-up. A sulphidic reactor is defined as conditions where dissolved oxygen is below 1 μM O₂ and sulphides are above 0.5 mM. We analyzed sulfur particles produced in continuous, long term lab-scale reactor experiments under various sulphide concentrations and sulphidic retention times. The analysis was performed with laser diffraction particle size analysis and light microscopy

Two identical lab-scale reactor set-ups were used with an absorber (A) having a liquid volume of 0.4 L and microaerophilic gas-lift reactor (C) having a liquid volume of 3.7 L (as shown in FIG. 1). Two additional reactor compartments could be added: a polysulphide reactor zone (B) having a liquid volume of 3.5 L between the absorber (A) and the microaerophilic gas-lift reactor (C) and a settler (D) having a liquid volume of 1.5 L after the microaerophilic gas-lift reactor.

A settler was included for the experiments with the highest H₂S loading rate to prevent sulphur accumulation in the system, i.e. to avoid operational issues such as foaming and clogging due to sulphur build-up. Experiments with lower H₂S loading rate were conducted without settler to collect a sample in which all particles were present that were produced under the specific experimental conditions, without removing any particles with the settler. The polysulphide reactor zone (B) is a zone with a retention time of reactor content (medium, microorganisms and sulphur particles) under anaerobic, (poly)sulphidic pressure. The presence of the polysulphide reactor zone (B) increases the Sulphidic′ Retention Time (SuRT).

The experiments carried out with the various conditions are numbered Examples 1-3 and Comparative Experiment A. An overview of the operational conditions per experiment is described in Table 1. The gas flow was recycled over the headspace of microaerophilic gas-lift reactor with a vacuum pump to prevent any release of H₂S gas and to reach low oxygen concentrations. The gas was introduced with a porous stone to the bottom of the inner column of the microaerophilic gas-lift reactor (C) to ensure proper oxygen transfer and mixing. Pure H₂S gas and oxygen were supplied by mass flow controllers. In case of pressure build-up, excess gas was discharged via a water-lock saturated with zinc acetate to capture any potentially present H₂S. The reactors were operated at 35° C. using a thermostat bath and climate-controlled cabinet.

TABLE 1

| | Comparative | | | |
|---|---|---|---|---|
| Process condition | Experiment A | Example 1 | Example 2 | Example 3 |
| Polysulphidic | no | yes | yes | yes |
| reactor zone (B) | | | | |
| Settler (D) | Yes | Yes | No | No |
| SuRT (min) | 4.8 | 45.0 | 38.7 | 38.7 |
| Total dissolved | 8.3 | 4.4 | 1.9 | 1.0 |
| sulphide in | | | | |
| polysulphidic | | | | |
| reactor | | | | |
| zone (mM) | | | | |
| S-loading rate | 4.4 | 4.4 | 2.0 | 1.0 |
| (g SL−1 | | | | |
| microaerophilic | | | | |
| reactor day−1) | | | | |
| Total system | 5.6 | 9.1 | 7.6 | 7.6 |
| volume a (L) | | | | |
| pH in reactor (C) | 8.4 | 8.5 | 8.7 | 8.5 |
| Duration (days) | 31 | 28 | 16 | 41 |

The medium consisted of a buffer with 6.6 g L⁻¹ Na₂CO₃ and 69.3 g L⁻¹ NaHCO₃ in demineralized water at pH 8.5. Fresh buffer was supplied at a constant flow to maintain enough alkalinity in the system. Furthermore, a nutrient stock was supplied for biological growth containing (in g per 1 L of demineralized water): K₂HPO₄, 0.1; MgCl₂ 6H₂O, 0.0203; NaCl, 0.6; CH₄N₂O, 0.06 and 2 mL L⁻¹ trace element solution as in Pfennig, N., Lippert, K. D., 1966. Uber das Vitamin B12-bedurfnis phototropher Schwefelbacterien. Arch. Microbiol. 55, 245-256.

Comparative Experiment A was inoculated with centrifuged microorganisms (to remove excess sulfur) from a lab-scale sulfur producing gas-lift bioreactor, operated under continuous conditions, like the conditions applied in these experiments. The original inoculum of this reactor was obtained from a well-characterized industrial scale Thiopaq process of applicant. To remove the sulfur, the reactor content was centrifuged at 4500 RPM for 20 minutes (using a FirLabO, Froilabo, Paris, France). A pellet was formed with two layers: a bottom layer of elemental sulfur and a pellet with microorganisms on top. The pellet with microorganisms was carefully washed off. Example 3 was inoculated with centrifuged microorganisms directly taken from the above mentioned Thiopaq process. Experiment 1 and 2 were inoculated with microorganism-rich process solution from Comparative Experiment A and Example 3.

Reactors (B,C) were filled with medium and inoculated. In all experiments, the set-up was operated in continuous mode without interruption. Throughout all experiments, the H₂S load was kept constant for that experiment. The H₂S load was used to set the total sulphide concentration in the polysulphide reactor zone. To keep the conversion efficiency from sulphide to sulfur high, the oxidation-reduction potential (ORP) was set at −360 mV vs. Ag/AgCl, which is a representative set-point for industrial reactors. The ORP set-point was controlled by a proportional-integral (PI) controller. The PI controller regulated the oxygen supply rate. Samples (well-mixed reactor content with sulfur particles, medium and micro-organisms) were taken for analysis at a sampling port in the middle of the polysulphide reactor zone (B) (Exp. 2 and 3) and the microaerophilic gas-lift reactor (C) (all experiments). The sampling tubes from the reactor were flushed three times prior to sampling to obtain a representative sample.

Reactors were equipped with sensors for temperature and ORP (Triple Junction, platinum rod, glass electrode equipped with an internal Ag/AgCl reference electrode, ProSense, Oosterhout, The Netherlands). The particle size distribution (PSD) is expressed both volumetrically and numerically; in a volumetric based particle size distribution, larger particles have a heavier weight as, due to their size, they often comprise a larger percentage of the total solid volume. In a numeric based distribution, each particle has an equal weight, independent of the particle size. According to common practice, when a PSD must be represented by a single value, the median (D50) of the PSD was reported to show the particle size development over time. The median has a better way of representing the central location of the data in a non-normal distribution than the mean.

The process selectivity for elemental sulfur was calculated by the mass balance based on the H₂S supply and measurement of dissolved sulfur products formed. The term ‘HS⁻’ is used to refer to the sum of total dissolved sulphide (H₂S, HS⁻ and S²⁻) as most of the dissolved sulphide is present as HS⁻ at pH 8.5.

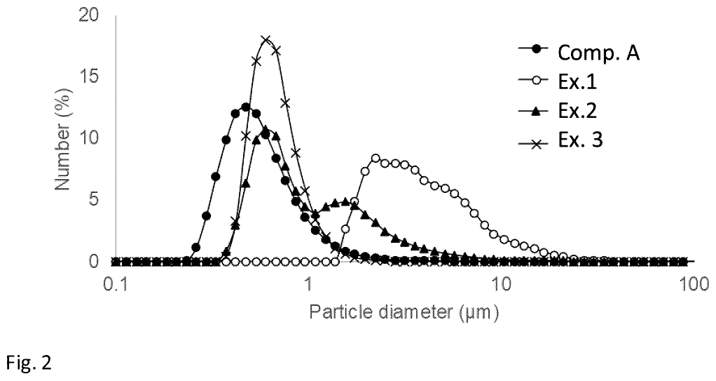

In Experiment A and in Examples 1, 2 and 3 sulphide was successfully converted to elemental sulfur, leading to the presence of sulfur particles in the reactor solution. Typical particle size distributions (PSD) of sulfur particle samples taken from the microaerophilic reactor during these experiments are shown in FIG. 2.

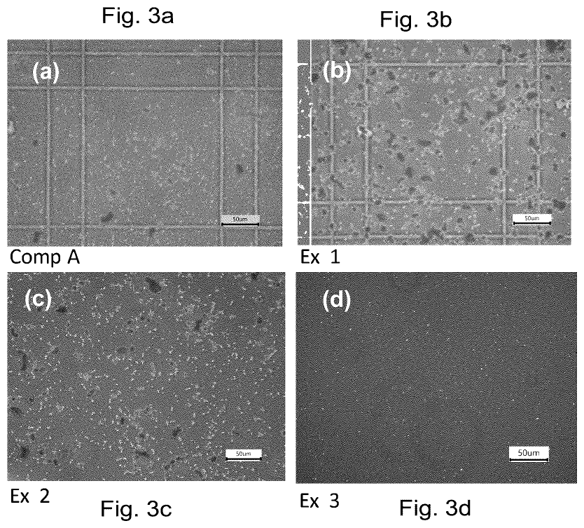

The sulfur particles formed under the various experimental conditions had distinctively different morphologies as observed with light microscopy as shown in FIG. 3. Sulfur particles can be distinguished in the light microscopy pictures as light, radiant particles (single particles) or darker patches with light, radiant edges (agglomerated particles). The black spots in the background are microorganisms.

In the pictures of comparative Experiment A many small individual (sub)micron-sized sulfur particles are visible, which is in good agreement with the particle size distribution shown in FIG. 2. In Comparative Experiment A also large agglomerates (˜20 μm) are present in seemingly low concentration. The concentration was likely too low to be visible in the number-based particle size distribution. In Comparative Experiment A the particles seem mainly globular (rough and smooth) and around a size of 1 μm.

In Example 1, however, small (sub)micron particles are hardly visible (FIG. 3(b)). Also, in Example 2 not a lot of these particles seem present, at least not as many as in Comparative Experiment A (FIG. 3(c)). Larger, agglomerated, sulfur particles can be observed in the pictures of Examples 1 and 2. In both examples, large agglomerates are visible of 20-30 μm, but also smaller ones of 5-10 μm. The center of the agglomerates appears dark due to the thickness of the sample, which could be observed while focusing the microscope. No microorganisms were observed attached to the agglomerates, as they could easily be distinguished as small black spots of around 1 μm in the sample. It is possible that due to mixing during PSD measurement, the larger aggregates were slightly broken down, and thus not measured. In addition, since the PSD is number based, the small particles weigh just as heavy in the distribution as the larger ones, and there are clearly more small particles than larger ones in terms of numbers. However, the larger ones are more visible in the microscopy picture. From the rough, lumpy edges of the agglomerates it can be observed that they are composed of many small constituent crystals.

FIG. 3 shows the influence of a polysulphide reactor zone on the formation of agglomerates. Further the light microscopy pictures show the smallest particles were hardly present in Example 1 and to only some extent in Examples 2 and 3.

The removal of the smallest particles in Examples 1, 2 and 3 is related to the formation of polysulphides in the polysulphide reactor zone. Polysulphides are yellow to orange and by the yellow color of the sulphidic reactor, it could be deduced that indeed polysulphides were formed.

The bisulphide content, polysulphide content, average chain length and the content of elemental sulphur as part of polysulphide was measured according to the method of this invention. It was found that these measurements fitted well to a mathematical model. The model inputs are the volume based average PSD of the four experiments and the operational conditions under which these particles were produced. From these PSDs, the volume fraction of particles with a diameter <1 μm was calculated. By multiplying this volume fraction with the average measured concentration of elemental sulfur in the experiments, the total concentration of particles <1 μm was calculated. Then, three outputs were calculated: the percentage of elemental sulphur (S₀) in that could be converted to polysulphide (Sₓ²⁻), the percentage equilibrium Sₓ²⁻ and the absolute content of elemental sulphur as part of polysulphide (S₀ in Sₓ²⁻), expressed in mM.

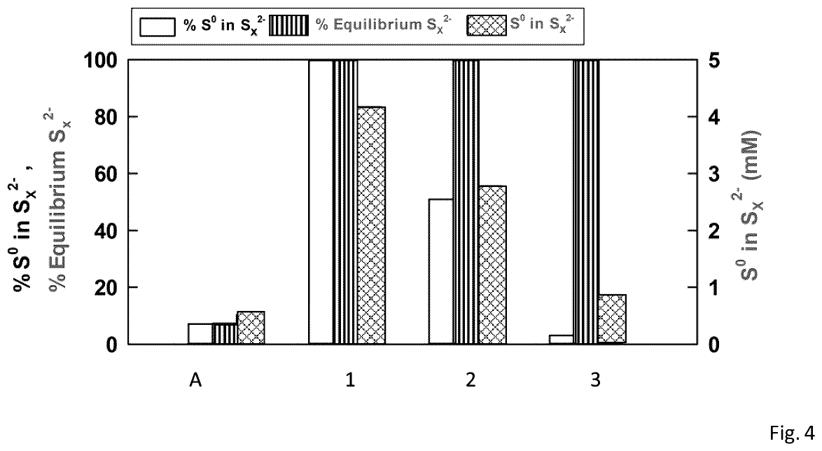

Our modelling results support the experimentally obtained findings that the smallest sulfur particles dissolve in the polysulphide reactor zone, due to polysulphide formation to the extent that the conditions allowed for. Equilibrium Sₓ²⁻ between sulfur, sulphide and polysulphide was reached for Examples 1, 2 and 3 (See FIG. 4). In Comparative Experiment A little polysulphide formation was expected due to the short SuRT, which agrees with the large amount of submicron particles found in the corresponding lab experiment. The modelling results support this. These particles would have been the ones most prone to react to polysulphide, due to their high surface-to-volume ratio.

These results from these experiments and models illustrate the invention: when the sulfur absorbing column is provided with reactors that promote the correct degree of mixing and residence time, and/or with higher starting sulphide concentrations, the higher (or total) equilibrium achieved between polysulphides and sulphides allow for the reacting-away of small elemental sulfur particles that were provided to the sulphidic chamber to form polysulphides. The resulting steady-state in the system (for example as measured in the bioreactor) is absent of smaller sulfur particles (<1 μm), and/or is concentrated in larger particles.

1. A process to continuously treat a hydrogen sulphide comprising gas, the process comprising:
(a) contacting the hydrogen sulphide comprising gas with an aqueous alkaline liquid comprising sulphide-oxidising bacteria and elemental sulphur particles thereby producing a loaded aqueous liquid comprising dissolved sulphide, polysulphide compounds, sulphide-oxidising bacteria and elemental sulphur particles and a gas having a lower content of hydrogen sulphide,
(b) passing the loaded aqueous liquid through a polysulphide reactor zone comprising one or more plug flow reactor zones,
(c) contacting the loaded aqueous liquid with an oxidant to enable the sulphide-oxidising bacteria to oxidise sulphide to elemental sulphur, thereby producing an enriched aqueous liquid comprising an increased amount of elemental sulphur particles, and
(d) separating elemental sulphur particles from the enriched aqueous liquid,
wherein residence time of the loaded aqueous liquid between its preparation in (a) and its supply to (c) is between 3 and 45 minutes, and
wherein content of elemental sulphur as part of the polysulphide compounds in the loaded aqueous liquid [S⁰ in Sₓ²⁻] as supplied to (c) is above 0.7 mM.

2. The process according to claim 1, wherein the content of elemental sulphur as part of the polysulphide compounds in the loaded aqueous liquid [S⁰ in Sₓ²⁻] as supplied to (c) is above 1 mM.

3. The process according to claim 1, wherein for a period of at least 1 week the daily average content of elemental sulphur as part of the polysulphide compounds in the loaded aqueous liquid [S⁰ in Sₓ²⁻] as supplied to (c) is above 0.7 mM.

4. The process according to claim 1, wherein the content of elemental sulphur as part of the polysulphide compounds in the loaded aqueous liquid meets the following condition:
$$[ {S}^{0} in {S}_{x}^{2 -} ] \geq 1.7 ⋆ [ {HS}^{-} ] * {10}^{( - 9.17 + pH )} ,$$
wherein [S₀ in Sₓ²⁻] is the content of elemental sulphur as part of the polysulphide compounds in the loaded aqueous liquid as supplied to (c) expressed in mM, [HS⁻] is the sulphide concentration expressed in mM of the loaded aqueous liquid as supplied to (c), and wherein pH is the pH of the loaded aqueous liquid as supplied to (c).

5. The process according to claim 4, wherein
[S⁰ in Sₓ²⁻]≥2.8*[HS⁻]*10^{(-9.17+pH)}.

6. The process according to claim 1, wherein the hydrogen sulphide comprising gas has a hydrogen sulphide content of between 0.1 and 3 vol. % and a carbon dioxide content of above 20 vol %.

7. The process according to claim 1, wherein the aqueous alkaline liquid is increased in temperature by indirect heat exchange with the loaded aqueous liquid and/or with an external heat source thereby obtaining a heated aqueous alkaline liquid which is used in (a).

8. The process according to claim 1, wherein (b) comprises passing the loaded aqueous liquid through a polysulfide reactor zone comprising one or more plug flow reactor zones, the polysulphide reactor zone having an upstream region and a downstream region.

9. The process according to claim 8, wherein part of the aqueous alkaline liquid comprising sulphide-oxidising bacteria is directly supplied to the upstream region of the polysulphide reactor zone.

10. The process according to claim 8, wherein in the polysulphide reactor zone part of the loaded aqueous liquid is recycled from the downstream region to the upstream region in the polysulphide reactor zone.

11. The process according to claim 10, wherein the part of the loaded aqueous liquid as isolated from the downstream region, before it is recycled to the upstream region in the polysulphide reactor zone, flows via a zone having a residence time of between 5 and 45 minutes.

12. The process according to claim 10, wherein the part of the loaded aqueous liquid which is recycled from the downstream region is increased in temperature before being recycled to the upstream region in the polysulphide reactor zone.

13. The process according to claim 1, wherein (a) is performed in a vertical column wherein continuously the hydrogen sulphide comprising gas is fed to the column at a lower position of the column and the aqueous liquid comprising sulphide-oxidising bacteria is continuously fed to a higher position of the column such that a substantially upward flowing gaseous stream contacts a substantially downwards flowing aqueous stream.

14. The process according to claim 13, wherein part of the aqueous liquid comprising sulphide-oxidising bacteria is continuously fed to a higher position of the column to contact with the upflowing gaseous stream in a first contacting zone which generates an intermediate loaded aqueous liquid and part of the aqueous liquid comprising sulphide-oxidising bacteria is continuously fed to an intermediate position of the column to contact together with the intermediate loaded aqueous liquid with the upflowing gaseous stream in a second contacting zone.

15. The process according to claim 1, wherein as part of (a) part of the aqueous liquid comprising sulphide-oxidising bacteria is (a1) continuously contacted in with the hydrogen sulphide comprising gas to obtain a first intermediate loaded aqueous liquid and an intermediate gas having a lower intermediate content of hydrogen sulphide,
wherein as part of (a) another part of the aqueous liquid comprising sulphide-oxidising bacteria is (a2) continuously contacted with the intermediate gas having a lower intermediate content of hydrogen sulphide to obtain a second intermediate loaded aqueous liquid and the gas having a lower content of hydrogen sulphide, and
wherein the first intermediate loaded aqueous liquid is combined with the second intermediate loaded aqueous liquid to obtain the loaded aqueous liquid.

16. The process according to claim 15, wherein each first and second intermediate loaded aqueous liquids flow through separate first and second polysulphide reactor zones respectively in which polysulphide reactor zones polysulphide compounds are formed by reaction of the dissolved sulphide and the elemental sulphur.

17. The process according to claim 16, wherein the first and second polysulphide reactor zones each comprise one or more plug flow reactor zones.

18. The process according to claim 16, wherein part of the first intermediate loaded aqueous liquid rich in polysulphides is supplied to the second polysulphide reactor zone to increase the polysulphide content in the second intermediate loaded aqueous liquid.

19. The process according to claim 18, wherein the first and second polysulphide reactor zones each comprise one or more plug flow reactor zones and wherein the part of the first intermediate loaded aqueous liquid rich in polysulphides is supplied to an upstream region of the second polysulphide reactor zone to increase the polysulphide content in the second intermediate loaded aqueous liquid.

20. The process according to claim 1, wherein at least a part of the enriched aqueous liquid of (c) is recirculated to (a).

21. A sulphur reclaiming process facility, comprising:
(a) an absorption column provided with an inlet for a hydrogen sulphide comprising gas, an outlet for a gas having a lower content of hydrogen sulphide at its upper end, an inlet for an aqueous alkaline liquid further comprising sulphide-oxidising bacteria and a first outlet for a loaded aqueous liquid at a lower elevation,
(b) a polysulphide reactor zone as part of the absorption column and positioned in the lower end of the absorption column and/or as part of a separate vessel, and
(c) an elemental sulphur recovery unit provided with an inlet fluidly connected to the aerobic bioreactor and provided with an outlet for elemental sulphur and an outlet for a liquid effluent poor in elemental sulphur,
wherein the polysulphide reactor zone comprises one or more plug flow reactor zones, the polysulphide reactor zone comprising an upstream end and a downstream end,
wherein the upstream end of the polysulphide reactor zone is fluidly connected to the first outlet for a loaded aqueous liquid,
wherein the downstream end of the polysulphide reactor zone is provided with a second outlet for the loaded aqueous liquid and with a recycle stream for part of the loaded aqueous liquid to the upstream end of the polysulphide reactor zone,
wherein the second outlet for the loaded aqueous liquid is fluidly connected to an aerobic bioreactor for oxidation of sulphide to elemental sulphur,
wherein the aerobic bioreactor is fluidly connected to the inlet for an aqueous alkaline liquid of the absorber column.

22. A sulphur reclaiming process facility, comprising:
(a) a first absorption column provided with an inlet for a hydrogen sulphide comprising gas, an outlet for an intermediate gas having a lower content of hydrogen sulphide at its upper end, an inlet for part of an aqueous alkaline liquid further comprising sulphide-oxidising bacteria and an outlet for a first intermediate loaded aqueous liquid at a lower elevation,
(b) a second absorption column provided with an inlet for the intermediate gas having a lower content of hydrogen sulphide, an outlet for a gas having a lower content of hydrogen sulphide at its upper end, an inlet for part of an aqueous alkaline liquid further comprising sulphide-oxidising bacteria and a outlet for a second intermediate loaded aqueous liquid at a lower elevation,
(c) a polysulphide reactor zone as part of the first absorption column and positioned in the lower end of the first absorption column and/or as part of a separate vessel,
(d) a polysulphide reactor zone as part of the second absorption column and positioned in the lower end of the second absorption column and/or as part of a separate vessel,
wherein the polysulphide reactor zones comprise plug flow zones, the sulphide reactor zones comprising an upstream end and a downstream end,
wherein the upstream end of the polysulphide reactor zone of the first absorption column is fluidly connected to the outlet for the first intermediate loaded aqueous liquid and the upstream end of the polysulphide reactor zone of the second absorption column is fluidly connected to the outlet for the second intermediate loaded aqueous liquid,
wherein the downstream end of the polysulphide reactor zone of the first absorption column is fluidly connected to the upstream end of the polysulphide reactor zone of the second absorption column,
wherein the downstream end of the polysulphide reactor zone of the second absorption column is fluidly connected to an aerobic bioreactor for oxidation of sulphide to elemental sulphur,
wherein the aerobic bioreactor is fluidly connected to the inlet for a part of the aqueous alkaline liquid of the first absorber column and fluidly connected to the inlet for a part of the aqueous alkaline liquid of the second absorber column, and
comprising an elemental sulphur recovery unit provided with an inlet fluidly connected to the aerobic bioreactor and provided with an outlet for elemental sulphur and an outlet for a liquid effluent poor in elemental sulphur.
